(19) United States
(12) Patent Application Publication
(10) Pub. No.: US 2002/0177190 A1
(43) Pub. Date: Nov. 28, 2002

(54) Recombinant simian GPR3 receptor

(76) Inventors: Yanbin Liang, Irvine, CA (US); David F. Woodward, Lake Forest, CA (US); Chen Li, Irvine, CA (US)

Correspondence Address: Carlos A Fisher Esq, Legal Department T2-7H, ALLERGAN INC, 2525 Dupont Drive, Irvine, CA, 92612, US

(21) Appl. No.: 09/956,470

(22) Filed: Sep. 18, 2001

Related U.S. Application Data
(63) Continuation-in-part of application No. 09/866,032, filed on May 24, 2001.

(51) Int. Cl. C07K 14/71; G01N 33/53; G01N 33/567; C07H 21/04; C12P 21/02; C12N 5/06

(52) U.S. Cl. 435/69.1; 435/320.1; 435/363; 530/350; 435/7.21; 536/23.5

(57) ABSTRACT

The invention provides recombinant materials for the production of the simian form of G-protein receptor 3 which differs in several aspects from the human counterpart. Compounds which interact with this receptor are useful in the treatment of glaucoma.

### TECHNICAL FIELD

This invention concerns recombinant materials for production of the simian G-protein receptor-3 (GPR3). This receptor is thus made available to identify ligands which are effective in treating hypertension, such as glaucoma.

### BACKGROUND ART

There is a plethora of receptors which participate in the regulation of biological processes that are activated through coupling to G-proteins and which effect signal transduction through this mechanism. This class of receptors, G-protein receptors (GPR) is involved in a wide variety of biological signaling including responses to opioids, thrombin, polyketides, and a variety of endogenous ligands. The G-proteins themselves are activated by phosphorylation of bound GDP to provide a GTP complexed form which activates adenylate cyclase and is returned to its basal state by hydrolysis of GTP to GDP, a reaction catalyzed by the G-protein itself. G-protein receptors generally have seven conserved hydrophobic transmembrane regions which connect hydrophilic loops extended extracellularly and intracellularly in alteration.

In some cases, receptors have been identified whose function is unknown. These “orphan” receptors, as well as receptors of known function, are useful in screening for compounds that agonize, partially agonize, or antagonize the signaling mediated by the receptor. The compounds thus identified have various therapeutic uses depending on the nature of the receptor itself.

For example, PCT application WO 00/21987 describes methods to identify compounds with regard to modulation of orphan receptors where the identified compounds are useful to treat Graves' disease and schizophrenia. WO 00/06597 describes a method for screening for compounds which are reactive as agonists or antagonists with G-protein coupled orphan receptor by contacting the candidate compounds with a fusion protein where the fusion protein comprises the receptor fused to a G-protein. WO 99/64436 describes recombinant production of a motilin receptor designated GPR38 which can be used to identify compounds that are useful in treating digestive disorders and bowel disease. EP 913471 describes the use of a G-protein receptor to screen for compounds that will be useful in diagnosing and treating HIV infection, anorexia and schizophrenia. WO 96/16087 describes a G-protein coupled receptor wherein compounds which agonize the receptor are said to be useful to treat asthma, Parkinson's disease, acute heart failure, hypertension, urinary retention and osteoporosis while antagonists are useful in treating hypertension, angina pectoris, myocardial infarction, ulcers, asthma, allergies, psychoses and a variety of eating disorders.

Most relevant to the recombinant materials and methods of the present invention is the disclosure of U.S. Pat. No. 5,998,164 which describes recombinant materials encoding various human G-protein coupled receptors, including GPR3. The disclosure of that document is incorporated herein by reference.

The above designated '164 patent shows the human form of GPR3 which is homologous to the simian form set forth herein. However, the simian form is distinct and as valuable as the human form in screening assays for identifying compounds that are useful in treating hypertension and especially hypertension associated with glaucoma.

### DISCLOSURE OF THE INVENTION

The invention resides in obtaining the nucleotide sequence encoding simian GPR3 which makes possible the recombinant production and display of GPR3, which can then be used in screening assays. The protein itself, recombinantly produced, may be used to generate antibodies which distinguish simian and human GPR3. The availability of the nucleotide sequence, also part of the present invention, permits production of recombinant materials which interrupt the production of simian GPR3 in primate or transgenic models of human disease. Antisense constructs and triple helix forming oligonucleotides are thus part of the present invention.

Thus, in one aspect, the invention is directed to an isolated nucleic acid comprising a nucleotide sequence which encodes simian GPR3, said GPR3 having an amino acid sequence as set forth in SEQ. ID. NO: 2. The preferred form of this isolated nucleic acid comprises the nucleotide sequence set forth in SEQ. ID. NO: 1 which is the natively occurring sequence. Also, within the scope of the invention are expression systems comprising the encoding nucleotide sequence which encodes the peptide set forth as SEQ. ID. NO: 2 operably linked to control sequences for expression. The invention also comprises nucleotide sequences complementary to that encoding the amino acid sequence of GPR3 especially complementary to SEQ. ID. NO: 1 of sufficient length to effect inhibition of expression of the endogenous sequence, as well as oligonucleotides which form triple helixes with the nucleic acids for production of GPR3 in situ.

In still other aspects, the invention is directed to recombinant host cells or transgenic animals or plants modified to contain a nucleotide sequence encoding simian GPR3 and methods for production of simian GPR3 which comprises culturing cells containing said nucleotide sequence or maintaining the transgenic animals and plants. The invention also comprises the recombinant GPR3 thus produced in isolated form. Also included within the scope of the invention are methods to identify compounds which are useful in the treatment of hypertension, and in particular glaucoma, by assessing the ability of these compounds to modulate the activity of GPR3. Also included within the scope of the invention are compounds thus identified and methods to treat glaucoma using these compounds. In addition, antibodies which are specific to simian GPR3 may conveniently be prepared.

### BRIEF DESCRIPTION OF THE DRAWINGS

FIG. 1 shows the nucleotide sequence of cDNA encoding simian GPR3 with the nucleotides which differ from the human sequence bolded.

FIG. 2 shows the deduced amino acid sequence with the differences from the human sequence indicated in bold.

### MODES OF CARRYING OUT THE INVENTION

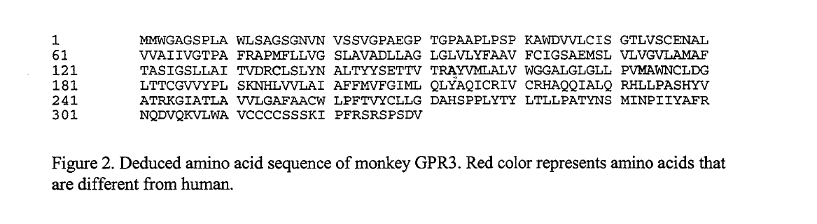

Simian GPR3 is a 330 amino acid protein with a calculated molecular mass of 34,967 daltons and is encoded by a 993 base pair open reading frame. The deduced amino acid sequence contains seven putative transmembrane domains characteristic of G-protein coupled receptors. These are underlined in FIG. 2. The availability of the recombinant materials for production of simian GPR3 is significant and basic to the invention.

Isolated nucleic acids which comprise nucleotide sequences encoding the simian GPR3 receptor are provided. By “isolated” is meant removed from the context in which the encoding nucleotide sequence normally occurs. Thus, the isolated nucleic acid may be in the form of a plasmid, integrated into a non-native genome, in purified form, contained in a heterologous cell, or in any other context wherein the nucleotide sequence is in unfamiliar surroundings. The relevant nucleotide sequence can be provided by recovery from its native environment using the procedures described hereinbelow, of can be synthesized using standard solid phase or solution phase nucleic acid synthesis techniques. These techniques are well-known in the are and can be effected using commercially available instrumentation.

The isolated nucleic acid comprising the encoding nucleotide sequence can then be manipulated using standard recombinant techniques to produce the GPR3 protein itself. The resulting protein can be displayed on the surface of appropriate host cells, in a manner most useful for screening compounds for their ability to modulate the receptor or can be obtained in purified form for the generation of antibodies or can be obtained, for example, as a fusion protein with a G-protein as described in WO 00/06597 cited above. The choice of hosts for production of the protein will depend on the purpose for its production. As set forth above, for use in screening assays, the receptor is preferably displayed at the surface of host cells which can permit facile assay for activation of the receptor. Such assay methods are generally known in the art, and include assessment for the formation of cyclic AMP, assays for signal transduction in general, and the like. Preferred cells for use in such assays are mammalian or vertebrate hosts, although in some circumstances yeast or procaryotic cells may be satisfactory. For such production, the encoding nucleotide sequence is typically coupled to control sequences, including promoters, ribosome binding sites, terminating sequences and so forth which are required for its expression. The choice of these control sequences is dependent on the nature of the host cell and a wide variety of such systems is commercially available. Alternatively, the nucleotide sequence encoding GPR3 may be integrated into the genetic complement of a host and employ the endogenous control sequences.

The encoding nucleotide sequence can also be integrated into the chromosomes of transgenic animals which can then be used as model systems for human or other animal conditions associated with overactivity or underactivity of GPR3. Thus, means are well understood to produce transgenic mice, for example, by inserting an expression system or just the nucleotide sequence encoding simian GPR3 into oocytes of embryonic stem cells and transplanting the modified genetic complement into blastocysts. Plants can also be transformed to contain the nucleotide sequence under conditions where the protein is produced.

The invention is also directed to screening methods for identifying compounds which modulate the activity of the receptor and thus identify compounds which are useful in the treatment of glaucoma and other conditions which are associated with hypertension. The compounds thus identified can then be further subjected to confirmatory assays which verify their ability to lower blood pressure. Such assays are well understood in the art. The successful compounds can then be formulated into pharmaceutical or veterinary compositions and used in methods to treat hypertension, including glaucoma. The formulations will be suitable for the condition to be treated, such as eye drops for the treatment of glaucoma, or oral or injectable formulations, transdermal formulations, transmucosal formulations and the like for treatment of systemic conditions, or indeed for the treatment of glaucoma per se.

The recombinant GPR3 produced can also be purified using known techniques, such as producing the recombinant protein with a histidine tag or FLAG sequence, thus permitting its chromatographic separation from other proteins, and thus used to elicit antibodies specific for the simian receptor. The resulting antibodies (which include antibody fragments, such as FAB fragments, or single chain forms such as Fv forms, or other immunospecific fragments) can be used to distinguish the simian GPR3 from the human form. The ability to make this distinction is useful in assessing levels of these receptors in transgenic animals, for instance. To obtain the antibodies, fragments of the protein could also be used (and perhaps conveniently synthesized chemically) which include portions where there are amino acid differences from the human receptor. Thus, elucidation of the coding sequence for the simian form permits identification of those regions of the molecule that would be useful in raising antibodies which distinguish human from simian GPR3.

The antibodies of the invention include monoclonal forms of the antibodies which are available using the techniques of Kohler and Milstein, for example, and recombinant forms of the relevant immunoreactive fragments. The fragments which are “immunospecific” are those which specifically interact with simian GPR3 but do not react with the human counterpart.

The elucidation of the nucleotide sequence encoding simian GPR3 also permits design of antisense sequences which can be used to modulate the levels of GPR3 produced in any animal model which produces GPR3 endogenously. Thus, the model may be the simian organism which ordinarily produces this receptor or may be a transgenic animal, such as a mouse, rabbit or rat which produces the receptor by virtue of its genetic modification. The antisense sequences can thus be used to regulate levels to explore the characteristics of conditions associated with hyperactivity or hypoactivity of this receptor. An alternative way to regulate the expression is the design of sequences which specifically interact with the double helix form to form triplexes. Sequence specific design in this context is also now well-known.

The following example is intended to illustrate but not to limit the invention.

### EXAMPLE 1

### Retrieval of cDNA Encoding Simian GPR3

Total RNA was isolated from cynomologus monkey ciliary body and iris following Qiagen RNA isolation kit protocol. Using oligo dT primer, the first strand cDNA was synthesized for Polymerase Chain Reaction (PCR) cloning. Two oligonucleotides based on human GPR3 receptor nucleotide sequence were designed and synthesized. The 5′ primer was (atgatgtggggtgcagg) (SEQ. ID. NO: 3) and 3′ primer was (ctagacatcactgggggt) (SEQ. ID. NO: 4). The PCR protocol for amplification, performed on Perkin Elmer 9700 using Taq polymerase and 20 ng of the first strand monkey ciliary cDNA as template, was as follows: denaturation at 94° C. for 30 s, annealing at 55° C. for 30 s, for 35 cycles. The 993 bp amplified product was subsequently isolated from a low-melt agarose gel and subcloned into PCRII vector, and sequenced.

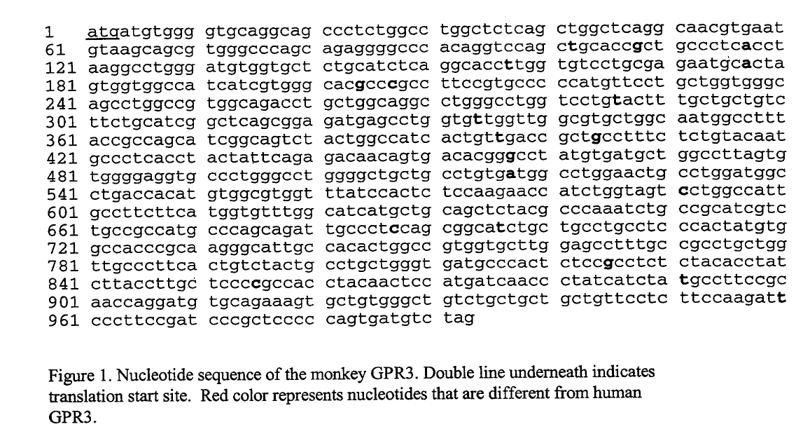

The nucleotide sequence obtained is shown in FIG. 1, and the deduced amino acid sequence is shown in FIG. 2.

1. An isolated nucleic acid molecule comprising a nucleotide sequence encoding simian GPR3 receptor having the amino acid sequence of SEQ. ID. NO: 2.

2. The nucleic acid of claim 1 wherein said nucleotide sequence has the sequence of SEQ. ID. NO: 1.

3. A nucleic acid molecule which comprises an expression system for simian GPR3 which expression system comprises a nucleotide sequence encoding SEQ. ID. No: 2 operably linked to control sequences for its expression.

4. Recombinant host cells comprising the expression system of claim 3.

5. A method to produce simian GPR3 which method comprises culturing the cells of claim 4 under conditions wherein said nucleotide sequence is expressed.

6. Recombinant simian GPR3 prepared by the method of claim 5.

7. The recombinant simian GPR3 of claim 6 which is displayed at the surface of recombinant host cells.

8. The simian GPR3 of claim 6 fused to G-protein.

9. A method to identify a compound useful in the treatment of hypertension which method comprises contacting the displayed GPR3 of claim 7 with said candidate compound and assessing the ability of said compound to modulate the activity of the receptor, whereby a compound which modulates the activity of said receptor is identified as useful in treating hypertension.

10. A method to identify a compound useful in the treatment of hypertension which method comprises contacting the displayed GPR3 of claim 8 with said candidate compound and assessing the ability of said compound to modulate the activity of the receptor, whereby a compound which modulates the activity of said receptor is identified as useful in treating hypertension.

11. A pharmaceutical composition containing the compound identified by the method of claim 9.

12. A pharmaceutical composition containing the compound identified by the method of claim 10.

13. A method to treat hypertension which method comprises administering to a subject in need of such treatment an effective amount of the pharmaceutical composition of claim 11.

14. A method to treat hypertension which method comprises administering to a subject in need of such treatment an effective amount of the pharmaceutical composition of claim 12.

15. The method of claim 13 wherein said hypertension is manifested as glaucoma.

16. The method of claim 14 wherein said hypertension is manifested as glaucoma.

17. A non-human, transgenic animal modified to contain the expression system of claim 3.

18. The animal of claim 17 wherein the control sequences are endogenous to said animal.

19. An isolated single stranded nucleic acid having a nucleotide sequence complementary to SEQ ID NO:1 or a sufficient portion thereof to inhibit expression.

20. A method to modulate the production of simian GPR3 which method comprises providing a subject which produces GPR3 with the single stranded nucleic acid of claim 19.

21. A single stranded nucleic acid which specifically forms a triplex with a nucleic acid duplex containing at least a portion of SEQ ID NO: 1 that is required for production of simian GPR3.

22. A method to modulate the production of simian GPR3 which method comprises providing a subject which produces GPR3 with the single stranded nucleic acid of claim 21.

23. Antibodies immunospecific for simian GPR3 of SEQ. ID. NO: 2.

24. A method to distinguish human GPR3 from simian GPR3 which method comprises contacting a sample suspected of containing either simian GPR3 or human GPR3 with the antibodies of claim 22 and detecting the formation of a complex between any simian GPR3 contained in said sample with said antibodies, thereby identifying GPR3 contained in said sample as simian GPR3.
